(19) United States
(12) Patent Application Publication
(10) Pub. No.: US 2011/0318777 A1
(43) Pub. Date: Dec. 29, 2011

(54) DEVICE FOR METERING CELLS, METHOD FOR METERING CELLS AND ALSO USE OF THE DEVICE

(75) Inventors: Jungtae Kim, Saarsbrucken (DE); Holger Krause, Neukirchen (DE); Jihwang Park, Saarbrucken (DE); Sebastian Beck, St. Ingbert (DE)

(73) Assignee: KIST-EUROPE FORSCHUNGSGESELLSCHAFT MBH, Saarbrucken (DE)

(21) Appl. No.: 13/157,102

(22) Filed: Jun. 9, 2011

(30) Foreign Application Priority Data
Jun. 9, 2010 (EP) 10 005 952.6

(51) Int. Cl. C12Q 1/06; C12M 1/34

(52) U.S. Cl. 435/39; 435/287.1

(57) ABSTRACT

The invention relates to a device for metering cells and also to a method for metering cells. Furthermore, the invention relates to the use of the device for metering cells.

### CROSS-REFERENCE TO RELATED APPLICATION

The present application claims the benefit of European Patent Application No. 10 005 952.6, filed Jun. 9, 2010, the disclosure of which is incorporated by reference in its entirety.

### BACKGROUND OF THE INVENTION

The invention relates to a device for metering cells and also to a method for metering cells. Furthermore, the invention relates to the use of the device for metering cells.

It is known that more than 10,000 screenings per day are possible by means of robot-assisted metering systems. In order to keep the costs for these tests low, minimisation of the sample volume is sought on the one hand and, on the other hand, the test methods are optimised. Exact meterings in the submicrolitre range thereby place serious demands on conventional metering devices for HTS applications (High Throughput Screening).

In the past, so-called cell-based assays (testing of media which contain cells) in medical research and bioscience were mainly implemented manually or with the help of simple devices since the application of an HTS system for cells is difficult. The exact volume and the precise number of cells in the samples is thereby important. A conventional HTS system cannot however control the number of cells but only the volume. With further development of cell-based series of experiments, a suitable HTS system is however currently required. There are already evaluation approaches on the market for cell-based series of experiments with 100 cells up to merely one cell.

Conventional HTS systems only control the volume of the sample. The result of the evaluation is however greatly dependent upon the number of cells in the sample, this dependence increasing with reducing numbers of cells in the sample.

A device with which drops can be metered is known from US 2008/0286751 A1. This device is provided with a first channel, a second channel, an intersection of these channels and also an opening at which the drop emerges.

In the case of conventional systems, the error probability during metering of volumes is at less than 2%, the associated error probability for the number of cells, in contrast, is above 10%. Furthermore, in the case of samples which contain only one cell, exact control of the number of cells is essential.

### Features of the Invention

Starting herefrom, it is an object of the present invention to make possible metering of cells with a low error probability both with respect to the volume of the drop and with respect to the number of cells in the drop.

This object is achieved by the characterizing features of the device for metering cells described herein, the method for metering cells, use of the device, and the advantageous embodiments and developments thereof.

The device according to the invention for metering cells comprises or consists of a basic body, there being provided, in at least one plane of the basic body, at least one inlet- and outlet channel for the carrier liquid, and also at least one cleaning channel and at least one further inlet channel for the metering medium essentially perpendicular to the channels disposed in this plane, and these channels opening into a cell collector module, the channels having at least respectively one valve and/or one diffuser structure, the cell collector module having a cell outlet with a metering valve and a control unit for the initiation of the metering process and a cell detection device being provided, the cell detection device being disposed on or at the basic body or being integrated in the basic body.

By means of the device according to the invention, metering of a controlled number of cells in one drop with a specific volume is possible. Furthermore, this drop contains hardly any impurities or undesired molecules since a backwash function and cleaning of the cell collector is made possible by the device according to the invention. Furthermore, the device according to the invention is not particularly susceptible to concentration variations of the cells in the carrier liquid.

The following designs are thereby possible as valves or microvalves in the inlet- and outlet channels: electromagnetically, piezoelectically or electrostatically actuated microvalves, ball microvalves, pressure-actuated valves.

The basic body of the device according to the invention can be polygonal, preferably rectangular. The basic body is preferably shaped such that use with a robot arm is possible in a simple manner, i.e. that the basic body has a shape which is easy to grip. The basic body can also be configured such that it can be securely connected to the receiving means on the robot arm, which can be effected for example by screwing. Furthermore, the cell collector module can be disposed in the centre.

The device preferably has at least one inlet channel for a by-pass flow which opens into the at least one inlet channel for the carrier liquid. By means of the by-pass flow, separation of the cells which are added through the inlet channel for the carrier liquid can be made possible.

In particular, the cell collector module can be constructed from a cell collector and a metering valve partial housing. These can be exchanged for example individually or as a module.

Preferably, the metering valve is constructed from a counterpart and a membrane. Undesired dripping of the device is hence avoided. Furthermore, such a metering valve is easy to clean.

The cell collector of the device according to the invention preferably has a membrane which is possibly elastic, and the metering valve partial housing has a counterpart which can be in particular conical, spherical or cylindrical, the membrane preferably abutting against the counterpart with pre-tension.

The metering valve partial housing, the membrane and/or the counterpart can be provided at least partially with a hydrophobic or hydrophilic coating. Furthermore, the metering valve partial housing, the membrane and/or the counterpart can be manufactured also from a hydrophobic or hydrophilic material. Consequently, better detachment of the drop is made possible, as a function of the media used. The use of hydrophobic or hydrophilic materials is dependent upon the application, i.e. for example what type of carrier liquid or metering medium is used.

Consequently, as a function of the cells to be metered (suspension culture, adherent cells) and the media used, a suitable coating or suitable material can be selected for the metering valve partial housing, the membrane or the counterpart.

The metering valve partial housing preferably has a valve cone as counterpart in the centre on its side orientated towards the cell collector and also 1 to 30 borings for the metering medium which are disposed outside a crescent-like groove for anchoring the cell collector structure.

The valve cone hereby closes the metering valve in cooperation with the elastic membrane which abuts against the valve cone with pre-tension provided that cells are collected or the device is cleaned. The crescent-like groove serves for stable anchoring of the cell collector structure. Furthermore, the crescent-like groove serves for stabilisation of the cell collector structure and furthermore as safety device against rotation between metering valve partial housing and cell collector.

Furthermore, the metering valve partial housing and/or the cell collector can be exchangeable in a modular fashion. Consequently, rapid exchange of the cell collector or of the metering valve can be effected, on the one hand, for example after contamination and, on the other hand, the device can be used consequently flexibly for the most varied of types of cells since other cell collectors are required respectively according to the cell size or cell type. For this purpose, cell collectors and metering valve partial housings with different geometries can be inserted in the basic body.

Furthermore, the geometry of the cell collector can determine the maximum number of cells to be metered via the radius of the filter- or cell collector structure. In this context, reference is also made to the possibility that the space between the cell collector structure and the counterpart to the membrane of the metering valve can be varied and hence influences a maximum number of cells to be metered. Since various cell types have different sizes, it is necessary to adapt the cell collector structures of the respective cell collectors to the cells, i.e. the spacings between the comb-like raised portions of the cell collector structure must fit respectively the cell type to be metered.

The cell collector module is preferably coated at least partially with at least one material, in particular non-adhesive coatings comprising or consisting of polyethyleneglycol (PEG), poly(2-hydroxyethylmethyl(meth)acrylate) (poly(HEMA)), silicone, polymethyl(meth)acrylate (PMMA), polyacrylamide, biocompatible coatings, in particular laminin, fibronectin, collagen I/III/IV, lysin or treated polystyrene. Possibly, an adhesive layer which is disposed for example between the cell collector and the non-adhesive coating can be required. Furthermore, a plasma treatment of the cell collector can be effected at least partially in addition to the coating or instead of a coating.

By means of these coatings, adherently growing cells can be prevented for example from remaining in the cell collector. Consequently, the coating or the plasma treatment serves for surface optimisation and hence for reducing an error probability with respect to the number of cells in the metered drop.

The basic body can have a safety device for prevention of rotation of the cell collector module in the basic body, by means of which rotation of the cell collector module relative to the basic body can be prevented. This improves in particular the seating of the cell collector and also of the metering valve partial housing, i.e. the cell collector module, in the basic body and furthermore enables optimum sealing of the system. In particular, the safety device for prevention of rotation can be configured such that mounting of the cell collector or of the cell collector module in the wrong position is impossible.

A method for metering cells is according to the invention, the carrier liquid with the cells to be metered flowing through the inlet channel for the carrier liquid in the direction of the cell collector, the number of cells being determined by means of the cell detection device, the cells accumulating in the cell collector module and at least a part of the carrier liquid discharging through the outlet channel for the carrier liquid, whilst the valve in the outlet channel for the carrier liquid is opened and the valve in the outlet channel for the cleaning is closed and, after achieving the number of cells to be metered, the control unit has the effect that a pulse is passed to the inlet channel of the metering medium and, before or simultaneously, the valve in the outlet channel for the carrier liquid and also the valve in the inlet channel for the carrier liquid is closed or the diffuser structure acts as passive valve, the valve in the outlet channel for the cleaning liquid remaining closed, the metering medium being added through the inlet channel for the metering medium and at least one cell being discharged in a drop.

Furthermore, the pressure wave or the pulse in the metering medium can be produced for example piezoelectrically or by an electrovalve or a magnetic valve. Also a mechanical production of the pressure wave or of the pulse would be conceivable.

The cells are hereby firstly counted and accumulated in the cell collector. If the desired number of cells is achieved, the cells in one drop are metered. The additional cleaning function makes it possible that the structure of the cell collector can be freed of cell residues and other impurities or, if more cells than desired have accumulated in the cell collector, these can be rinsed out of the cell collector.

Furthermore, a further medium which serves for separating the cells can be added through the inlet channel for the by-pass flow and effects an arrangement of the cells in succession in the inlet channel for the carrier liquid.

As a result, improved counting of the cells by the cell detection device subsequently to be passed through is made possible. The cell detection device can detect the throughflowing cells for example via an optical measurement, impedance measurement or capacitance measurement and convey the result to the control unit. Furthermore, the distance of the cell detection device from the cell collector and also the flow rate are known so that it can be calculated by the control unit how many cells are situated at what time in the cell collector. This process is termed cell collection.

If the desired number of cells in the cell collector is achieved, then the control unit initiates the metering process. For this purpose, the outlet channel for the carrier liquid is closed by a valve and a pulse is passed to the inlet channel for the metering medium, as a result of which the cells present in the cell collector are discharged by the metering valve. The volume of the metering liquid can thereby be regulated so that a drop with a controlled volume and a controlled number of cells is produced. The diffuser structure or the valve in the inlet channel for the carrier liquid thereby acts similarly to a non-return valve and prevents cells from being able to move from the cell collector structure back into the inlet channel for the carrier liquid or the pressure wave continuing too strongly in the inlet channel for the carrier liquid. Normally, the cells are discharged in a mixture of carrier liquid, metering medium and possibly liquid from the by-pass flow.

It is however also possible that the carrier medium or the liquid are removed or discharge out of the by-pass flow, if required, before metering of the metering medium, for example through the outlet channel for the carrier liquid.

After the metering, the outlet channel for the carrier liquid is opened again and the cell collector can be filled again with the desired number of cells.

Subsequently and/or during the metering process, provided that the number of cells has been exceeded, a cleaning step can be implemented, cleaning liquid being introduced through the inlet channel for the metering medium and the valve of the outlet channel for the carrier liquid and also the metering valve being closed and the liquid discharging through the outlet channel for the cleaning liquid. The cleaning liquid can also be identical to the metering medium or the carrier liquid.

A cleaning step can be required for example when the cell collector or the cell collector module is to be freed of impurities such as cell residues or proteins, and also in order to remove cells from the cell collector if too many cells have accumulated there. For the cleaning, the outlet channel for the carrier liquid is closed and the cleaning channel is opened. Then a liquid, in particular a cleaning liquid, is conducted through the inlet channel for the metering medium, which liquid allows for example residues to discharge from the cell collector structure through the cleaning channel.

For the method according to the invention, there can be used as carrier liquid, cell culture medium, salt solutions, buffered solutions, isotonic common salt solution, 2(4-(2-hydroxyethyl)-1-piperazinyl)-ethanesulphonic acid (HEPES)-buffered solutions, tris(hydroxymethyl)-aminomethane (TRIS)-buffered solutions, solutions containing sodium citrate, solutions containing trypsin, solutions containing ethylenediaminetetraacetic acid (EDTA), solutions containing antibiotics, solutions containing antimycotics or mixtures hereof.

There are used preferably as metering medium, cell culture medium, salt solutions, isotonic common salt solution, 2(4-(2-hydroxyethyl)-1-piperazinyl)-ethanesulphonic acid (HEPES)-buffered solutions, tris(hydroxymethyl)-aminomethane (TRIS)-buffered solutions, solutions containing fluorescence markers, solutions containing 3-(4,5-dimethylthiazol-2-yl)-2,5-diphenyltetrazoliumbromide (MTT) or mixtures hereof.

Buffered salt solutions (balanced salt solutions) are used as
in conjunction with carbohydrates (glucose), the main energy source for the metabolism of the cells is provided;
transport- or diluting media for maintaining the osmotic balance;
provision of the cells with water and specific inorganic ions, essential for normal cell metabolism;
buffer system for obtaining the physiological pH range.

Common salt solutions are:
Dulbecco's Phosphate Buffered Saline
Earle's Balanced Salts
Hanks' Balanced Salts
Tyrode's Salts
Phosphate Buffered Saline
   and also many specific modifications of these solutions (often with the addition of “modified”) and more rarely used buffered salt solutions (Alsever's Solution, Gey's Balanced Salt Solution, Kreb's Henseleit Buffer Modified, etc.).

Standard cell culture media are:

Common Media
Medium 199: standard tissue-culture medium; provided one of the first completely defined nutrient sources for the cell culture;
DMEM (Dulbecco's Modified Eagle's Medium): Modification of the Basal Medium Eagle (BME) for use with embryonic mouse cells: great number of further modifications for specific applications/special cell lines;
MEM (Minimum Essential Medium Eagle): widely used synthetic cell culture medium with many variations/modifications to the original formula for use in various applications/cell types;
RPMI-1640 (Roswell Park Memorial Institute): a bicarbonate buffer system, enriched with vitamins and amino acids, frequently used for the culture of primary cells, mainly leucocytes; however used nowadays for cultivation of many cell lines,
   and also many further special or more rarely used media, such as: Ames' Media, BGJb Medium (Fitton-Jackson Modification), Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium and William's Medium E.

Special Media

Further-developed media with lower serum- or glutamine use or formulations modified for special applications. In this context, the following cell culture medium should be regarded by way of example only since there are also many further compositions and media here.
MCDB Medium (Ham et al.): low-protein- and low-serum medium with specific formulation coordinated to individual cell lines (embryonic cultures, etc.)

Serum-Free Media
Chemically-defined media and supplements which can be used without the addition of animal supplements, such as serum (FCS etc.). The constituents are often coordinated to specific cell lines and have a restricted field of application.

Known cell lines for which serum-free media have been developed are: CHO cells (Chinese Hamster Ovary), hybridoma systems and many more.

Further Culture Media for Microorganisms, Plants and Fungi

Dependent upon the type and group of microorganisms (yeasts, bacteria, fungi), there is a vast number of types of media and media formulations.

The most well-known formulation for a medium for the culture of bacteria is thereby LB medium.

For the method according to the invention, identical solutions can be used as carrier liquid and as metering medium. Furthermore, a solution identical to the carrier liquid and/or to the metering medium can be used as cleaning liquid. The selection of the mentioned solutions, media etc. should thereby be regarded by way of example since there is in addition still a large number of further usable solutions and media.

However, it is also possible that different solutions are used as carrier liquid and as metering medium. This is particularly of importance when for example cell-damaging supplements require to be used. If these are added to the metering medium, the cells are consequently brought in contact with these toxic supplements for a significantly shorter time interval, in comparison to the case in which these supplements are already contained in the carrier liquid.

Furthermore, the metered addition of specific substances can also be of interest when, from the beginning of the metered addition of the metering medium and from a specific substrate, kinetics, for example with respect to metabolisation of the substrate, are intended to be established.

According to the invention, the device is used for metering human cells, animal cells, procaryotes, eucaryotes and/or plant cells.

There should be mentioned here by way of example:

Primary Cell Lines:
All cells which have been removed directly from the human or animal body. They can often be cultivated only for a few passes.
Included herein are: lymphoid cells (white blood cells), hepatocytes (liver cells), etc.

They can be divided roughly into adherent lines (tissue-forming) and suspension cells (often blood cells), more than 90% of all primary cell lines being adherent.

Immortalised Cell Lines
These cell lines grow and multiply infinitely in vitro as long as the right culture conditions are maintained. There are included herein also, but not exclusively, carcinogenic cells and tumour cells.
There are included herein: HeLa (adherent, non-carcinogenic), MCF-7 (adherent, carcinogenic), JURKAT (suspension, carcinogenic), etc.
If other model organisms (Mus musculus, Danio rerio) are used, an analogous classification is undertaken. Unicellular organisms are excluded therefrom.
Low eucaryotes (e.g. fungi), Saccharomyces cerevisae, etc.), procaryotes (Escherichia coli, Staphylococcus aureus, etc.) but also many plant cells can be used analogously.

### BRIEF DESCRIPTION OF THE DRAWINGS

The subject according to the application is intended to be explained in more detail with reference to the subsequent FIGS. 1 to 11, without restricting said subject to these variants.

There are shown

FIG. 1 an isometric view of the upper side of the device according to the invention;

FIG. 2 an isometric view of the underside of the cell metering device;

FIG. 3A the device according to the invention in a view from below;

FIG. 3B the device in a side view from the right;

FIG. 3C the device in front view;

FIG. 3D the device in side view from the left;

FIG. 3E the device in rear view;

FIG. 3F the device in a view from above;

FIG. 4A a front view of the device;

FIG. 4B a horizontal section through the device during the cell collection process;

FIG. 5A the front view of the device;

FIG. 5B the horizontal section through the device during the metering process;

FIG. 5C an enlarged cut-out of FIG. 5B;

FIG. 6A the front view of the device according to the invention;

FIG. 6B a horizontal section through the device according to the invention during the cleaning process;

FIG. 7A a front view of the device according to the invention;

FIG. 7B a vertical section through the device according to the invention;

FIG. 8 the cell collector in various views;

FIG. 8A the cell collector in a view from diagonally above;

FIG. 8B the cell collector in a view from diagonally below;

FIG. 8C the cell collector in a view from above;

FIG. 8D a horizontal section through FIG. 8C;

FIG. 8E an enlarged cut-out of FIG. 8D;

FIG. 9 the metering valve partial housing in various views;

FIG. 9A the metering valve partial housing in a view from diagonally above;

FIG. 9B the metering valve partial housing in a view from diagonally below;

FIG. 9C the metering valve partial housing in a view from below;

FIG. 9D a side view of FIG. 9C;

FIG. 9E a view from above onto the metering valve partial housing;

FIG. 10 a view of the basic body from diagonally below without cell collector and metering valve partial housing;

FIG. 11 various views of the combination of cell collector and metering valve partial housing as cell collector module;

FIG. 11A a view from above onto a metering valve partial housing which is connected to a cell collector;

FIG. 11B a vertical section through FIG. 11A;

and

FIG. 11C shows a horizontal section through FIG. 11A.

### DETAILED DESCRIPTION

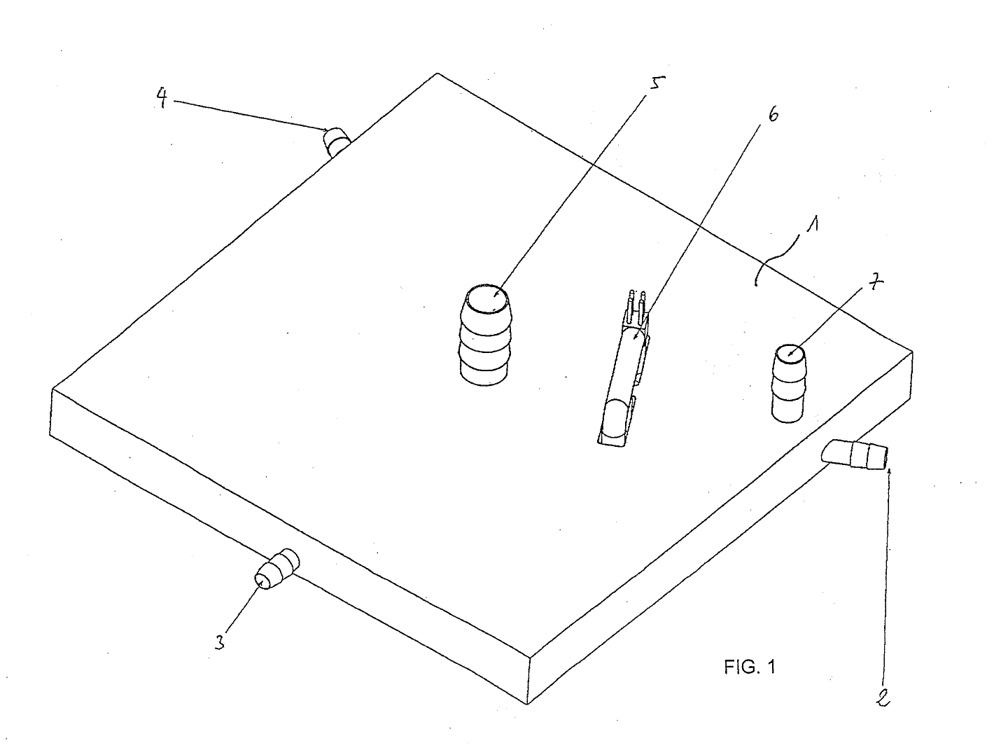

In FIG. 1, a view from diagonally above onto the device according to the invention is represented. The basic body 1 has an inlet channel for the metering medium 5 and also a cell detection device 6 on the upper side of said basic body. The inlet channel for the by-pass flow 7 is disposed perpendicular to the inlet channel for the carrier liquid 2. On the oppositely situated side of the inlet channel for the carrier liquid 2, the outlet of the cleaning channel 4 is disposed. The outlet channel for the carrier liquid 3 is disposed on the abutting surface.

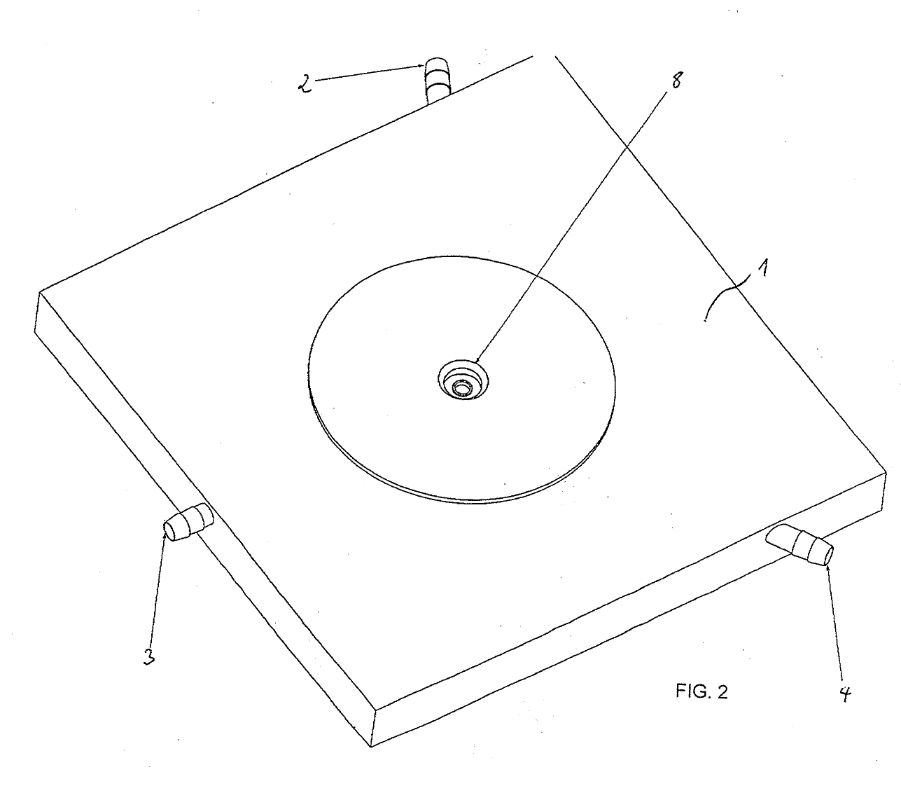

FIG. 2 shows a view from diagonally below. The outlet for the metered cells 8 is disposed in the centre of the basic body 1. The inlet channel for the carrier liquid 2 is situated opposite the outlet of the cleaning channel 4. On the side situated therebetween, the outlet channel for the carrier liquid 3 is disposed.

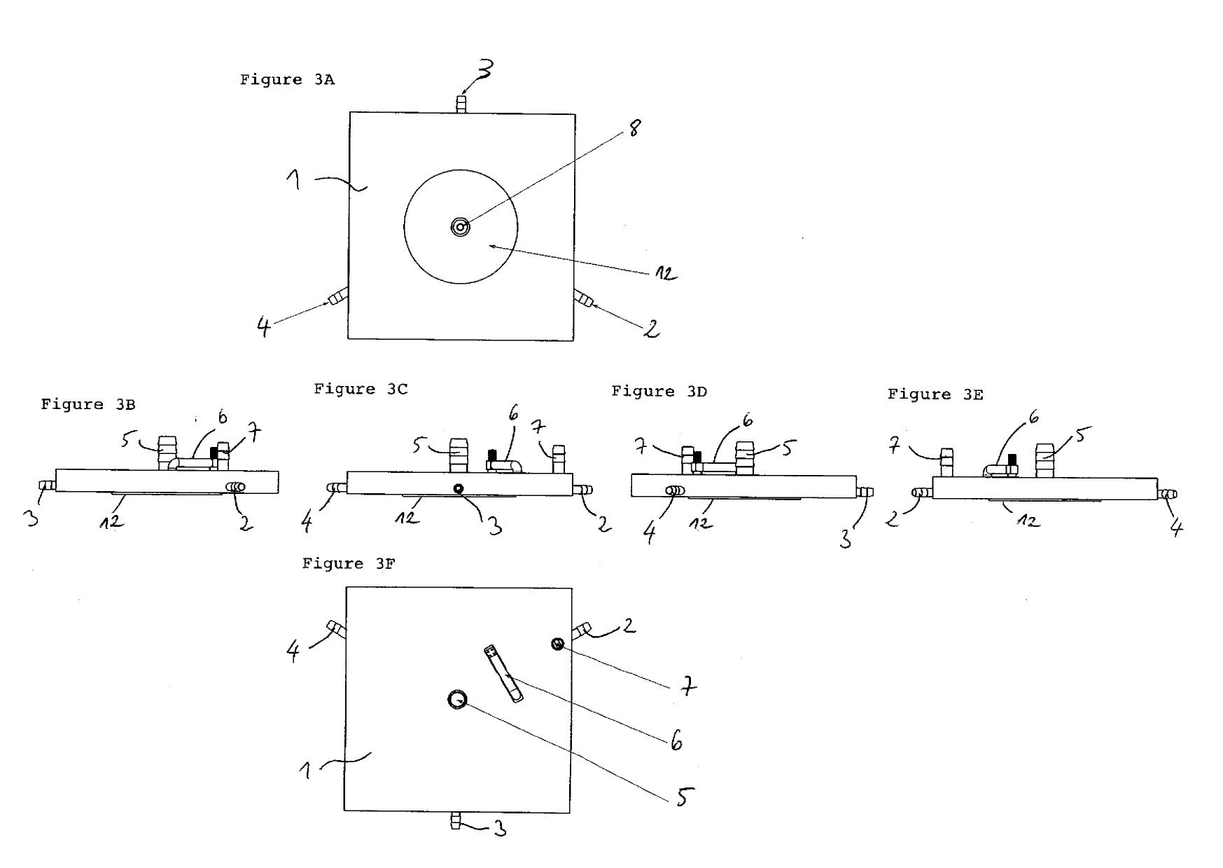

FIG. 3A shows a view from below of the device according to the invention for metering cells. The inlet channel for the carrier liquid 2, the outlet channel for the carrier liquid 3 and also the cleaning channel 4 open in the centre into the cell collector 13 which is not represented in this Figure. The outlet for the metered cells 8 is disposed in the centre in the retaining plate of the cell collector module 12 in the basic body 1. The cell collector module 23 consists of the cell collector 13 and the metering valve housing 14.

FIG. 3B shows the side view from the right. On the upper side of the basic body 1, the inlet channel for the metering medium 5, the cell detection device 6 and also the inlet channel for the by-pass flow 7 are disposed. On the underside of the basic body 1, the retaining plate for the cell collector module 12 is disposed. The outlet channel for the carrier liquid 3 points to the left. The inlet channel for the carrier liquid carrier 2 points forwards in this view.

In FIG. 3C, a front view of the device according to the invention for metering cells is represented. On the upper side of the basic body 1, the inlet channel for the metering medium 5, the cell detection device 6 and also the inlet for the by-pass flow 7 are disposed. The cleaning channel outlet 4 points to the left, the outlet channel for the carrier liquid 3 forwards and the inlet channel for the carrier liquid 2 to the right. The retaining plate for the cell collector module 12 is disposed below the basic body 1.

FIG. 3D shows a side view from the left. The inlet channel for the by-pass flow 7, the cell detection device 6 and also the inlet channel for the metering medium 5 are disposed on the upper side of the basic body 1. The outlet of the cleaning channel 4 points forwards and the outlet channel for the carrier liquid 3 to the right. The retaining plate for the cell collector module 12 is disposed on the underside of the basic body 1.

FIG. 3E shows a rear view of the device according to the invention. The inlet channel for the by-pass flow 7, the cell detection device 6 and also the inlet channel for the metering medium 5 are disposed on the upper side of the basic body 1. The inlet channel for the carrier liquid 2 points to the left and the cleaning channel 4 to the right. On the underside of the basic body 1, the retaining plate for the cell collector module 12 is disposed.

In FIG. 3F, a view from above onto the device is represented. The inlet channel for the metering medium (5) is disposed on the basic body (1) in the centre. The inlet channel for the carrier liquid 2, the outlet channel for the carrier liquid 3 and also the cleaning channel 4 open into it. Perpendicular to the inlet channel for the carrier liquid 2, the inlet channel for the by-pass flow 7 is disposed. Furthermore, the device according to the invention has a cell detection device 6 on the upper side of the basic body.

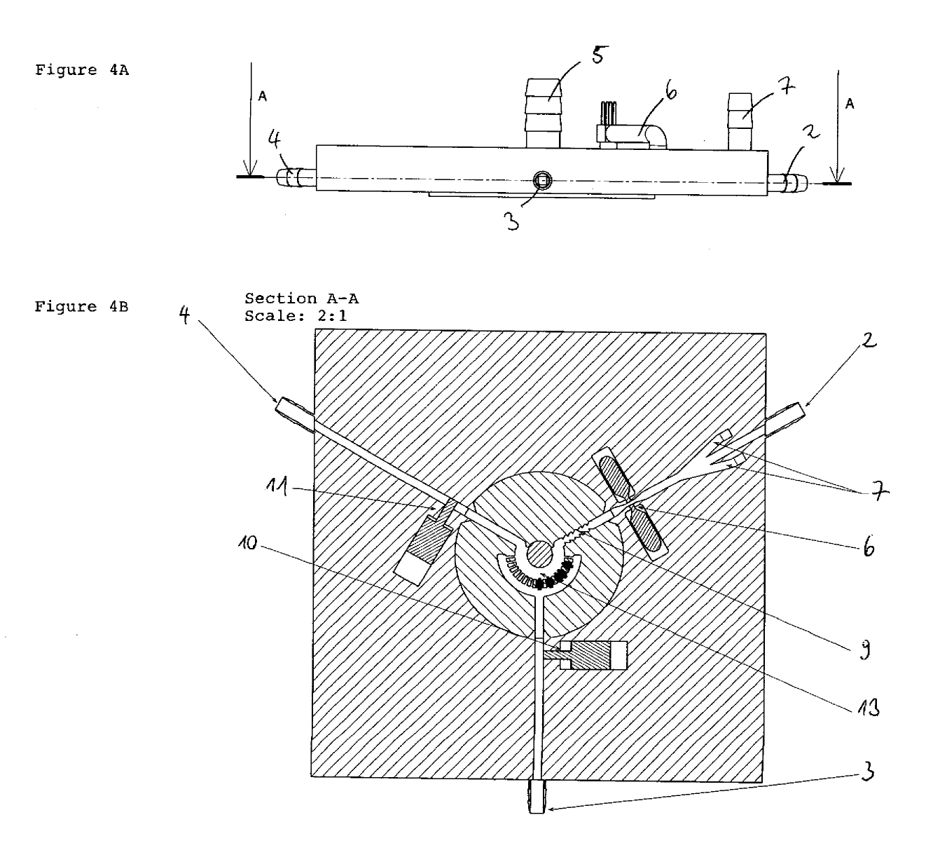

In FIG. 4A, a side view of the device according to the invention is represented. The inlet channel for the metering medium 5, the cell detection device 6 and also the inlet channel for the by-pass flow 7 are disposed on the upper side of the basic body 1. The cleaning channel 4 points to the left, the outlet channel for the carrier liquid 3 forwards and the inlet channel for the carrier liquid 2 to the right.

In FIG. 4B, a horizontal section through FIG. 4A is represented. This shows the valve positions during the cell collection process. By means of the inlet channel for the carrier liquid 2, the cells 13 are rinsed into the cell collector. The valve in the outlet channel for the cleaning liquid 11 is thereby closed and the valve in the outlet channel for the carrier liquid 10 is opened. The cell detection device 6 is disposed between the inlet for the carrier liquid 2 and the cell collector 13. Furthermore, inlet channels for the by-pass flow 7 which open into the inlet channel for the carrier liquid 2 are represented.

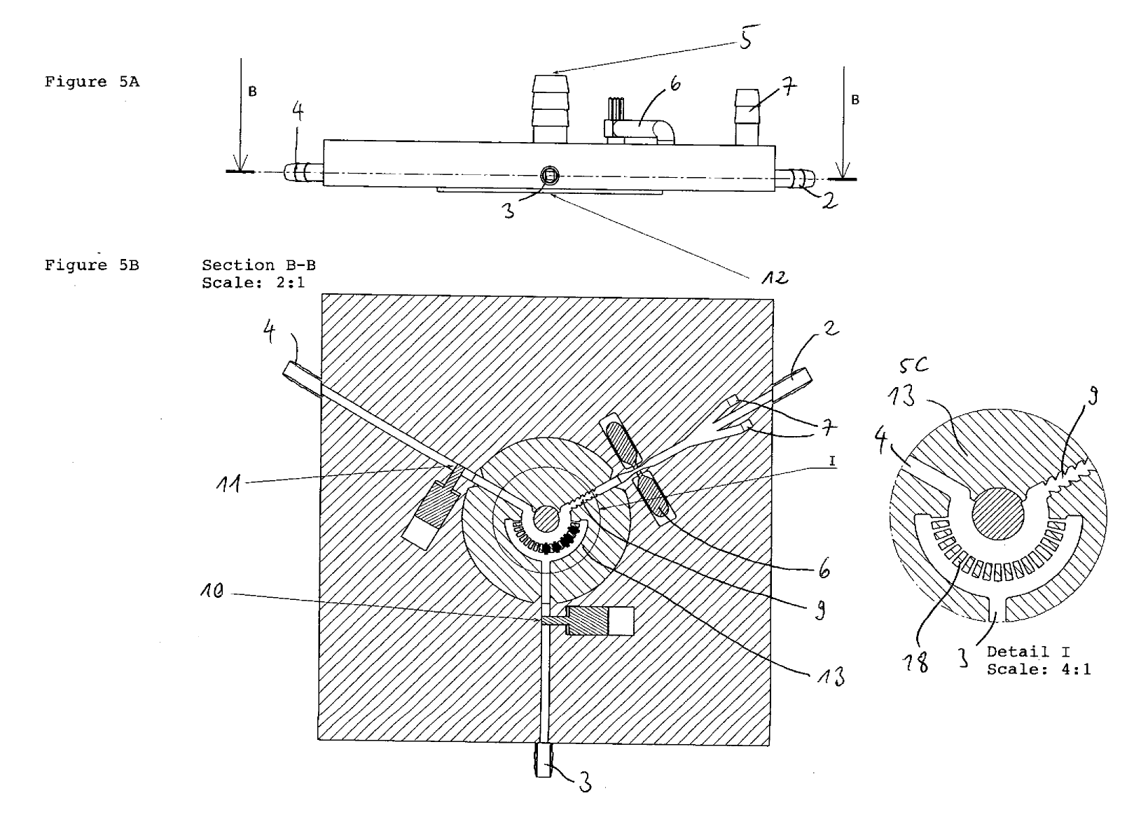

FIG. 5A shows a side view of the device according to the invention. The inlet channel for the metering medium 5, the cell detection device 6 and also the inlet channel for the by-pass flow 7 are disposed on the upper side of the basic body 1. The outlet of the cleaning channel 4 points to the left, the outlet channel for the carrier liquid 3 forwards and the inlet channel for the carrier liquid 2 to the right.

FIG. 5B shows a horizontal section through FIG. 5A during the metering process. Both the valve in the outlet channel for the cleaning 11 and also the valve in the outlet channel for the carrier liquid 10 are hereby closed. The diffuser structure 9 is an integral component of the cell collector 13 which is disposed adjacent to the cell collector structure 18. Counting of the cells is effected via the cell detection device 6 which is disposed on the inlet channel for the carrier liquid 2. Medium for separating the cells can be added through the inlet channel for the by-pass flow 7.

In FIG. 5C, a cut-out of the cell collector module 23 is represented enlarged. The latter has a cell collector structure 18. The outlet channel for the carrier liquid 3 is represented pointing downwards. The inlet channel for the carrier liquid 2 which opens into the cell collector 13 via the diffuser structure 9 points to the right. The outlet for the cleaning channel 4 points to the left.

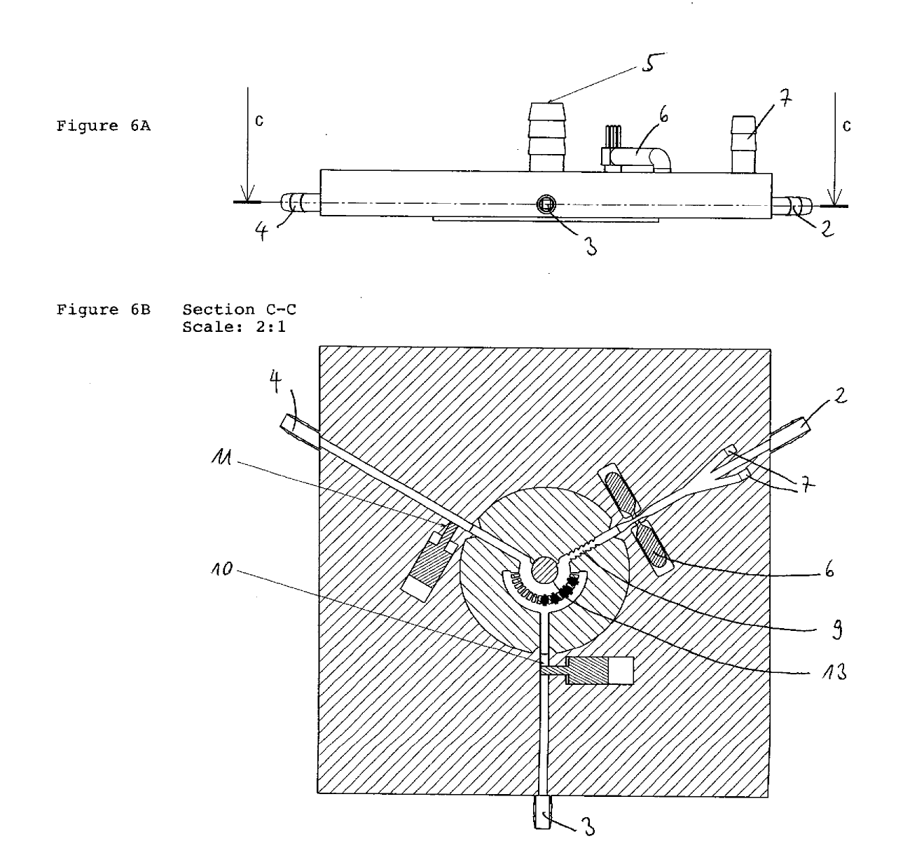

In FIG. 6A, a side view of the device according to the invention is represented. The inlet channel for the metering medium 5, the cell detection device 6 and also the inlet channel for the by-pass flow 7 are disposed on the upper side of the basic body 1. The cleaning channel outlet 4 points to the left, the outlet channel for the carrier liquid 3 forwards and the inlet channel for the carrier liquid 2 to the right.

In FIG. 6B, the horizontal section through FIG. 6A during the cleaning process is represented. The valve in the outlet channel for the carrier liquid 10 is thereby closed and the valve in the outlet channel for the cleaning 11 is opened. During the cleaning, the cleaning medium is rinsed through the inlet channel for the metering medium 5 into the cell collector 13 and, after it has passed the cell collector 13, is discharged via the cleaning channel 4.

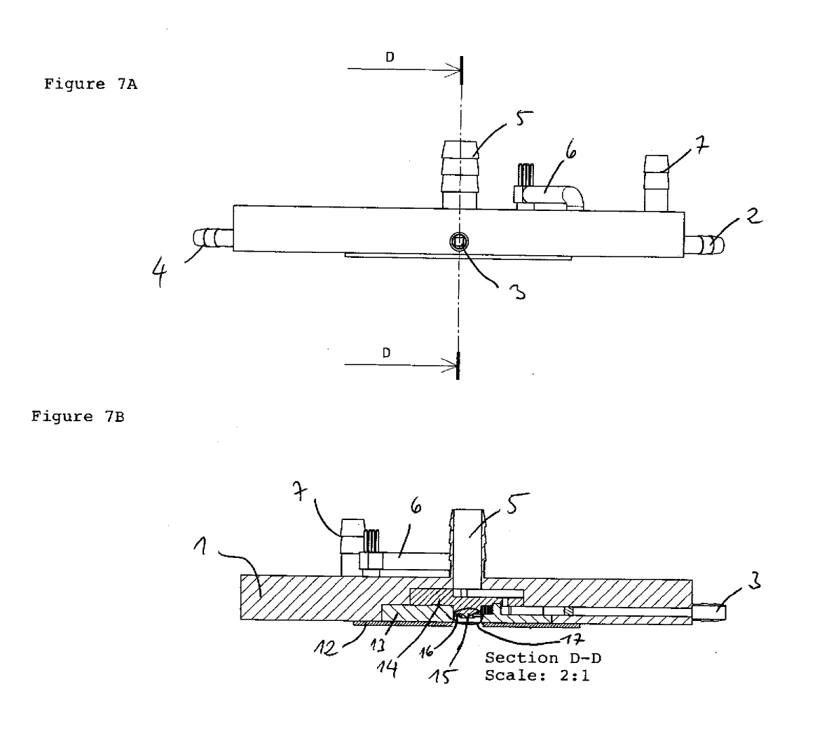

FIG. 7A shows a side view of the metering device according to the invention. The inlet channel for the metering medium 5, the cell detection device 6 and also the inlet channel for the by-pass flow 7 are disposed on the upper side of the basic body 1. The cleaning channel 4 points to the left, the outlet channel for the carrier liquid 3 forwards and the inlet channel for the carrier liquid 2 to the right.

FIG. 7B shows the vertical section through FIG. 7A. The outlet channel for the carrier liquid 3 hereby points to the right. Below the inlet channel for the metering medium 5, the metering valve partial housing 14 which has a valve cone as counterpart 15 is disposed. The membrane 16 is integrated in the cell collector 13. The membrane 16 abuts against the valve cone 15. It is normally raised from the valve cone 15 only in the case of the metering process by the pressure wave after the pulse to the inlet channel 5 so that the drop can be detached. The cell collector 13 which abuts directly against the metering valve partial housing 14 is fixed on the basic body 1 via the retaining plate of the cell collector module 12. The cell collector module is constructed from the cell collector 13 and the metering valve partial housing 14. The metering valve 17 is composed of the valve cone 15 and the membrane 16.

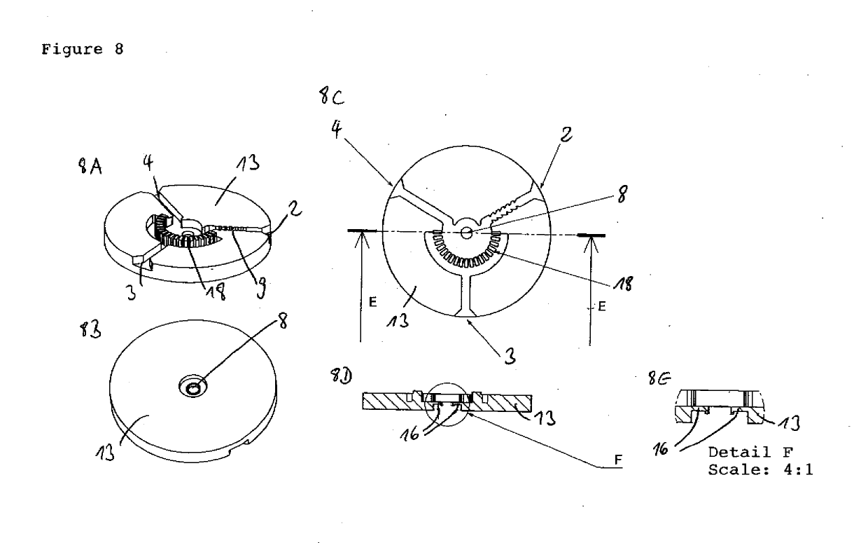

FIG. 8 shows various views of the cell collector.

In FIG. 8A, a view from diagonally above is represented. The inlet channel for the carrier liquid 2 hereby points to the right and the outlet channel for the carrier liquid 3 to the left at the front. The outlet for the cleaning channel 4 points to the left at the back. The cell collector structure 18 is represented in the centre of the cell collector 13.

FIG. 8B shows a view from diagonally below. The outlet for the metered cells 8 is disposed in the centre of the cell collector 13.

FIG. 8C shows a view of the cell collector 13 from above. The cell outlet 8 is represented in the centre of the cell collector 13. Furthermore, the cell collector structure 18 is disposed in the shape of a semicircle. The inlet channel for the carrier liquid 2 points to the right at the top, the outlet channel for the carrier liquid 3 downwards and the outlet of the cleaning channel 4 to the left at the top.

FIG. 8D shows a horizontal section through FIG. 8C. The membrane 16 which is integrated in the cell collector 13 is hereby represented in the centre of the cell collector 13.

FIG. 8E shows the marked cut-out F of FIG. 8D. The membrane 16 integrated in the cell collector 13 is represented here enlarged.

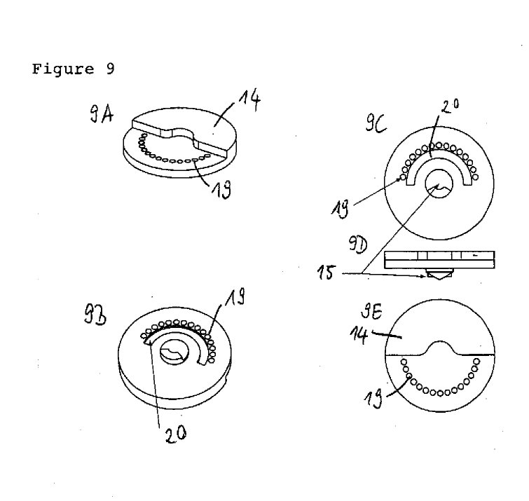

FIG. 9 shows various views of the metering valve partial housing 14.

In FIG. 9A, a view from diagonally above is represented. The metering valve partial housing 14 has borings for the metering medium 19.

In FIG. 9B, the metering valve partial housing 14 according to the invention is represented in a view from diagonally below. The borings for the metering medium 19 are detectable here and also the crescent-shaped groove 20 which serves for anchoring the cell collector structure 18.

In FIG. 9C, a view of the metering valve partial housing 14 is represented from below. The borings for the metering medium 19 abut directly against the crescent-shaped groove 20 for anchoring the cell collector structure 18. The valve cone 15 is represented in the centre.

FIG. 9D shows a side view of FIG. 9C. The valve cone 15 is situated in the centre on the lower side of the metering valve partial housing 14.

FIG. 9E shows the metering valve partial housing 14 in a view from above. Again the borings for the metering medium 19 can be detected here.

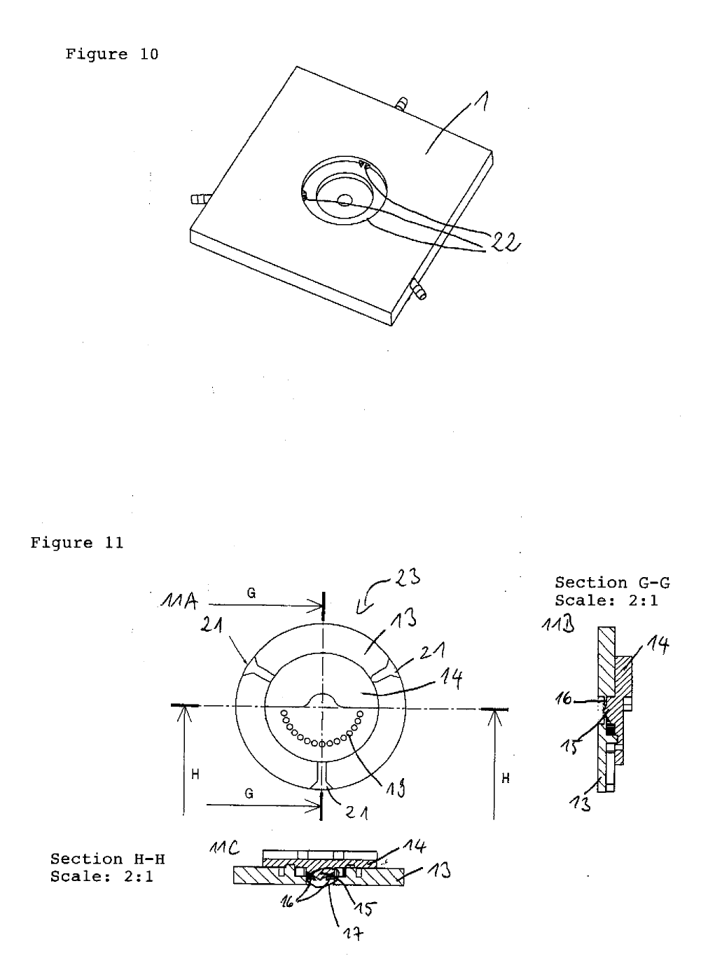

FIG. 10 shows the basic body 1 in a view from diagonally below. The recess for the cell collector module 23 made of the cell collector 13 and metering valve 14 is represented here. The safety device against rotation 22 serves for stable fixing of the cell collector 13 in the basic body 1.

FIG. 11 shows the metering valve partial housing 14 and the cell collector 13 in the assembled form as cell collector module 23.

In FIG. 11A, a view from above is represented. The cell collector 14 which has borings for the metering medium 19 is disposed on the cell collector 13. The cell collector 13 has three recesses 21 for the safety device against rotation 22. In this way, a stable mounting of the cell collector 13 and also of the cell collector module 23 in the basic body 1 is ensured.

FIG. 11B shows a vertical section through the cell collector module 23 represented in FIG. 11A. The metering valve partial housing 14 represented on the right has a valve cone as counterpart 15 which abuts directly against the membrane 16. Furthermore, the cell collector 13, which is connected to the metering valve partial housing 14, is represented.

FIG. 11C shows a horizontal section through FIG. 11A. The metering valve partial housing 14, which has a valve cone 15, is disposed above the cell collector 13. The membrane 16 is represented in the centre of the cell collector 13. The metering valve 17 consists of the membrane 16 integrated in the cell collector 13 and the valve cone as counterpart 15.

1. A device for metering cells comprising a basic body, there being provided, in at least one plane of the basic body, at least one inlet and outlet channel for the carrier liquid, and also at least one cleaning channel and at least one further inlet channel for the metering medium essentially perpendicular to the channels disposed in this plane, and these channels opening into a cell collector module, the cell collector module having a cell outlet with a metering valve and a control unit for the initiation of the metering process and a cell detection device being provided, the cell detection device being disposed on or at the basic body or being integrated in the basic body.

2. The device according to claim 1,
wherein the channels have at least respectively one valve and/or one diffuser structure.

3. The device according to claim 1,
wherein the basic body is polygonal, preferably rectangular, and the cell collector module is disposed in the centre.

4. The device according to claim 1,
wherein the device has at least one inlet channel for a by-pass flow which opens into the at least one inlet channel for the carrier liquid.

5. The device according to claim 1,
wherein the cell collector module is constructed from a cell collector and a metering valve partial housing.

6. The device according to claim 1,
wherein the metering valve is constructed from a counterpart and a membrane.

7. The device according to claim 6,
wherein the cell collector has a membrane which is possibly elastic, and the metering valve partial housing has a counterpart which is in particular conical, spherical or cylindrical, the membrane preferably abutting against the counterpart with pre-tension.

8. The device according to claim 6,
wherein the metering valve partial housing, the membrane and/or the counterpart are provided at least partially with a hydrophobic or hydrophilic coating, and/or are manufactured from a hydrophobic or hydrophilic material.

9. The device according to claim 5,
wherein the metering valve partial housing has a valve cone as counterpart in the centre on its side orientated towards the cell collector and also 1 to 30 borings for the metering medium which are disposed outside a crescent-like groove for anchoring the cell collector structure.

10. The device according to claim 5,
wherein the metering valve partial housing and/or the cell collector are exchangeable in a modular fashion.

11. The device according to claim 5,
wherein the cell collector module is coated at least partially with at least one material, in particular non-adhesive coatings comprising or consisting of polyethyleneglycol (PEG), poly(2-hydroxyethylmethyl(meth)acrylate) (poly(HEMA)), silicone, polymethyl(meth)acrylate (PMMA), polyacrylamide, biocompatible coatings, in particular laminin, fibronectin, collagen I/III/IV, lysin, treated polystyrene, and/or a plasma treatment of the cell collector is effected at least partially.

12. The device according to claim 1,
wherein the basic body has a safety device for prevention of rotation of the cell collector module in the basic body.

13. A method of metering cells by means of a device according to claim 1,
wherein the carrier liquid with the cells to be metered flows through the inlet channel for the carrier liquid in the direction of the cell collector the number of cells being determined by means of the cell detection device, the cells accumulating in the cell collector module and at least a part of the carrier liquid discharging through the outlet channel for the carrier liquid, whilst the valve in the outlet channel for the carrier liquid is opened and the valve in the outlet channel for the cleaning is closed and, after achieving the number of cells to be metered, the control unit has the effect that a pulse is passed to the inlet channel of the metering medium and, before or simultaneously, the valve in the outlet channel for the carrier liquid is closed and also the diffuser structure acts as passive valve in the inlet channel for the carrier liquid, the valve in the outlet channel for the cleaning liquid remaining closed, the metering medium being added through the inlet channel for the metering medium and at least one cell being discharged in a drop.

14. The method according to the preceding claim,
wherein a further medium which serves for separating the cells is added through the inlet channel for the by-pass flow and effects an arrangement of the cells in succession in the inlet channel for the carrier liquid.

15. The method according to claim 13,
wherein, subsequently and/or during the metering process, provided that the number of cells has been exceeded, a cleaning step is implemented, cleaning liquid being introduced through the inlet channel for the metering medium and the valve of the outlet channel for the carrier liquid and also the metering valve being closed and the liquid discharging through the outlet channel for the cleaning liquid.

16. The method according to claim 13,
wherein there are used as carrier liquid, cell culture medium, salt solutions, buffered solutions, isotonic common salt solution, 2(4-(2-hydroxyethyl)-1-piperazinyl)-ethanesulphonic acid (HEPES)-buffered solutions, tris(hydroxymethyl)-aminomethane (TRIS)-buffered solutions, solutions containing sodium citrate, solutions containing trypsin, solutions containing ethylenediaminetetraacetic acid (EDTA), solutions containing antibiotics, solutions containing antimycotics or mixtures hereof.

17. The method according to claim 13,
wherein there are used as metering medium, cell culture medium, salt solutions, isotonic common salt solution, 2(4-(2-hydroxyethyl)-1-piperazinyl)-ethanesulphonic acid (HEPES)-buffered solutions, tris(hydroxymethyl)-aminomethane (TRIS)-buffered solutions, solutions containing fluorescence markers, solutions containing 3-(4,5-dimethylthiazol-2-yl)-2,5-diphenyltetrazoliumbromide (MTT) or mixtures hereof.

18. The method according to claim 13,
wherein identical solutions are used as carrier liquid and as metering medium.

19. The method according to claim 13,
wherein different solutions are used as carrier liquid and as metering medium.

20. The method of claim 13, wherein the cells are human cells, animal cells, procaryotes, eucaryotes and/or plant cells.
